(19) United States
(12) Patent Application Publication
(10) Pub. No.: US 2019/0336633 A1
(43) Pub. Date: Nov. 7, 2019

(54) METHOD FOR PREVENTING DEPOSITION OF CONTAMINATION ON CERTAIN PARTS OF INSTRUMENTS PLACED IN A STEAM AUTOCLAVE DURING THE STERILIZATION PROCESS.

(71) Applicants: Maarten Antoon Nicolaas den Hartog, Arnhem (NL); Pamela Christine Musch, Arnhem (NL)

(72) Inventors: Maarten Antoon Nicolaas den Hartog, Arnhem (NL); Pamela Christine Musch, Arnhem (NL)

(21) Appl. No.: 16/465,210

(22) PCT Filed: Nov. 22, 2017
(86) PCT No.: PCT/NL2017/000019

§ 371 Date: May 30, 2019

(30) Foreign Application Priority Data
Dec. 2, 2016 (NL) 1042175

(51) Int. Cl. A61L 2/26; A61L 2/07

(52) U.S. Cl. CPC A61L 2/26; A61B 1/247; A61L 2202/24; A61L 2/07

(57) ABSTRACT

Method for preventing deposition of contamination on certain parts of instruments placed in a steam autoclave during the sterilization process, characterized in that the surface of an instrument that is meant to be kept clean, is covered and placed in the autoclave, and whereby by means of the method of attachment and or shape of the cover, the inner side of the cover facing the surface to be kept clean is held at a specific distance from said surface during the autoclaving process, with a limit value between a minimum of 0.2 mm and a maximum of 0.5 mm.

In a steam autoclave, during use it is inevitable that calcium residues and or residues of composite or corrosion will accumulate, originating from instrumentation sterilized in said autoclave. This pollution dissolves in steam and is spread with that steam through the entire autoclave and ends up during the condensation phase, via the precipitating water droplets, partly on the surface of the instrumentation, and adheres during the drying phase to said instrumentation. This is particularly a problem, where it involves high-quality specular surfaces, such as dental mouth mirrors with a dielectric mirror layer.

The specific problem of dirt deposition on high-quality specular surfaces after autoclaving has not been found in other patent applications.

U.S. Pat. No. 5,707,553 describes the operation of the autoclaving process and generally reports corrosion during the autoclaving process and the deposition of rust spots as a result on instruments placed in an autoclave. Here, a solution is proposed to make an addition to the water in order to prevent rust and spotting.

This solution has as the disadvantage of intervention in the autoclaving process with possible detrimental consequences and in any case it offers no remedy against the deposition of other particles than corrosion in the autoclave such as composite and lime or other consumed material.

Noteworthy in connection with the invention is U.S. Pat. No. 4,294,356. This application relates to an ‘anti-scratch protector case’; a facility with a semi-circular space above the mirror that protects the mirror surface from scratches through touch of other instruments. This application does not relate to preventing pollution from depositing on the instruments, but merely to providing a protection against damage. The solution offered does not offer an implicit solution to the problem of deposition of dirt residues because in the space above the mirror surface dirt can penetrate with the steam and can be deposit on the mirror surface by condensation and thus can still adhere to it during the drying stage.

Furthermore in this respect, the invention U.S. Pat. No. 6,048,503 is interesting, in which a closed container with small holes is proposed for the purpose of efficiently sterilizing instruments, and in addition the known solution of the use of a bag of vapor-permeable foil or plastic or gauze which goal is to keep the instruments sterile after the sterilization process; where the methods offered do not offer either a solution for the prevention of deposition of debris on the mirror surface, since here too steam can penetrate with dissolved pollution and, as stated in said patent, condenses and drips from the instrumentation onto a hot plate, from which it turns out that the instruments are getting wet and that dirt with that moisture will settle on the instruments.

None of the abovementioned patents or methods is intended to provide a solution for the deposition of dirt residues originating from lime, composite or other consumed materials on the mirror surface and in practice it appears that in the described solutions the aforementioned problem continues to occur and that in cases where residues of lime or composite or other consumed material dissolve in the steam, it can pass through the material of the packaging and, together with the steam, end up on the mirror surface and can still be deposited there as water droplets, so that deposition of dirt on the mirror surface remains possible.

It is the objective of the invention to provide a method whereby spot formation on the surface of the instrumentation or certain parts thereof due to deposition of dirt residues such as lime, composite and other consumed materials during the sterilization process in a steam autoclave is prevented. The here proposed method to reach the above objective is characterized in that the surface of an instrument to be kept clean is covered with in any case a water-drop-resistant material and is to be placed in the autoclave, further comprising a method of attachment and or the shape of said cover that is keeping the inner side of the cover facing the surface that is to be kept clean during the autoclaving process at a specific distance from said surface, with a limit value between minimum 0.2 mm and maximum 0.5 mm. This ensures that during the sterilization the amount of steam necessary for sterilization can enter through the opening between the above-mentioned surfaces, but during the condensation phase no water droplets can penetrate through the openings in the space between the two surfaces, while within that space itself too small an amount of water molecules is present to form water droplets. Those limit values of 0.2 mm and 0.5 mm are the result of tests based on the hypothetical invention, which have shown that the invention meets the objective in any case with the materials used stainless steel and plastic within aforementioned margins. The protective material can be formed in a dimensionally stable shape or it can be clamped in a dimensionally stable shape by use of a construction.

In case a surface to be protected is not flat, the inner side of said cover can be formed negatively towards the surface that is to be covered in parallel. Said cover can either be mounted directly on the instrumentation such, that the inflow of steam into the space between the cover and the surface of the instrumentation is not obstructed, or said cover can be mounted on a container in which the instruments are placed, for example in the form of a lid, or said cover can be rigidly attached to said container.

The distance that is to be maintained between aforementioned surfaces can also be ensured by providing the inner side of said cover with spacers, either supporting on the instrument or at the edge of the surface that is to be kept clean, and said distance is given a length of the desired specific distance between said surfaces. Materials that are suitable for this application include amongst others stainless steels and plastics resistant to temperatures of at least 121 degrees Celsius or, depending on the type of autoclave possible up to 134 degrees Celsius. Various types of instruments can be protected in the above described way. Especially with high-quality polished surfaces, such as mirror surfaces for dental applications, the advantage of this facility is evident. The method can be applied by means of specially designed products, such as a frame with water-dropping foil stretched onto said frame, provided with fastening means for accurate positioning.

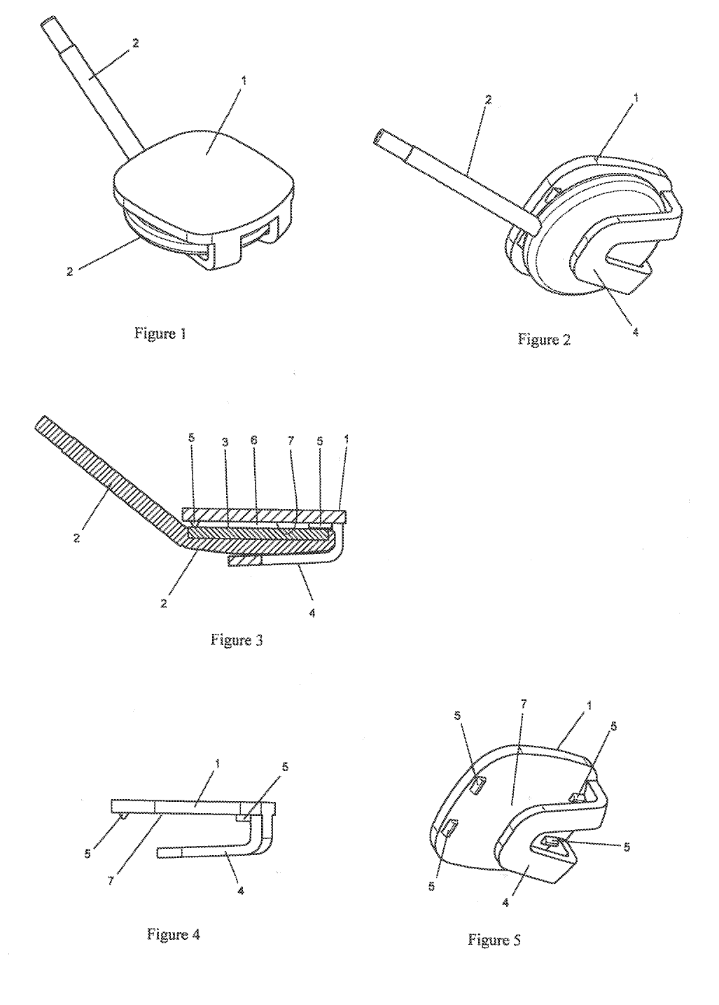

A preferred embodiment of such a product concerns a U-shaped object, the upper part of which serves to cover the surface that is to be protected which, to that end, on the side facing the surface that is to be covered, is formed negatively, parallel to the surface that is to be covered, and which at the edge is provided with spacers, in order to support at the edge of said surface and which part, together with the connecting bridge and the spacers forms a clamp, by means of which the product can be clamped onto the instrumentation, as will be described below with reference to FIGS. 1-6.

FIG. 1 and FIG. 2 show the overlap [1] with the clamp [4] mounted on mirror [2].

FIG. 3 shows the composition in longitudinal section, where the cover [1] is placed at a fixed position on the instrumentation [2], the clamp [4] holding the cover in place and the spacers [5] ensuring a space [6] to be created between the inner side [7] of the cover [1] and the surface [3] that is to be protected, of between 0.2 and 0.5 mm maximum. The facility is produced from a sufficiently rigid material to ensure that the space [6] remains between 0.2 and 0.5 mm.

FIG. 4 shows the cover [1] in side view with the spacers [5] and the clamp [4] and the inside of the cover (7).

FIG. 5 shows the cover [1] in bottom view with the spacers [5] and the clamp [4] and the inner side of the cover (7).

I claim:

1. Method for preventing deposition of contamination on instruments or on certain surfaces of said instruments placed in an autoclave during the sterilization process, characterized in that said instrument or part of said instrument in any case is covered during the sterilization process, with a water-resistant cover, further comprising a manner of attachment and or shape of said cover that is keeping the inner side of said cover facing the surface that is to be kept clean at a specific distance from said surface, whereby a limit value of at least 0.2 mm and a maximum of 0.5 mm between said inner side and said surface is maintained and further comprising that the space between said surfaces is kept accessible by openings laterally of said surfaces for the purpose of passage of steam.

2. Device for applying the method according to claim 1, further comprising said cover to be of a water-drop-resistant material and further comprising said cover to be provided with fastening means for mounting said cover at a guaranteed distance between 0.2 and 0.5 mm of the object or part of said object that is to be kept clean.

3. Device as claimed in claim 2, further comprising that on the inner side of said cover, facing said object that is to be kept clean, spacers are formed of a suitable length in order to preserve the distance between 0.2 and 0.5 mm to said object or a part thereof.

4. Device as claimed in claim 3, further comprising said inner side of said cover that is to be placed over said other object is formed negative in advance with respect to the surface of said object onto which it is mounted.

5. Device according to claim 2 or 3 or 4, further comprising said cover to be attached to said instrumentation.

6. Device as claimed in claim 2 or 3, further comprising said cover to be fastened to said instrumentation by means of a resilient clamp, engaging said instrumentation.

7. Device as claimed in claim 2 or 3 or 4 or 8, further comprising said cover to be mounted onto an instrument container, in which said object is held in a forced position.

8. Device as claimed in claim 2, further comprising said cover to be produced from stainless steel or high-quality heat-resistant plastic or a combination of both materials.
